(19) United States
(12) Patent Application Publication
(10) Pub. No.: US 2009/0297578 A1
(43) Pub. Date: Dec. 3, 2009

(54) BIOSOLUBLE COATING COMPRISING ANTI-PROLIFERATIVE AND ANTI-INFLAMMATORY AGENT COMBINATION FOR TREATMENT OF VASCULAR DISORDERS

(76) Inventors: MIKAEL O. TROLLSAS, San Jose, CA (US); Michael H. Ngo, San Jose, CA (US); Florencia Lim, Union City, CA (US); Syed F.A. Hossainy, Fremont, CA (US); Bozena Zofia Maslanka, Aptos, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 12/132,535

(22) Filed: Jun. 3, 2008

(51) Int. Cl. A61F 2/00; A61K 31/445; A61K 31/56

(52) U.S. Cl. 424/423; 514/326; 514/171

(57) ABSTRACT

Drug-delivery systems such as drug-delivery stents having an anti-proliferative agent such as everolimus and an anti-flammatory agent such as clobetasol are provided. Also disclosed are methods of treating a vascular impairment such as restenosis or vulnerable plaque.

### BACKGROUND OF THE INVENTION

1. Field of the Invention

This invention generally relates to a drug combination including an anti-proliferative drug such as everolimus and an anti-inflammatory agent such as clobetasol or dexamethasone for the treatment of a disorder such as restenosis and vulnerable plaque.

2. Description of the Background

Percutaneous coronary intervention (PCI) is a procedure for treating heart disease. A catheter assembly having a balloon portion is introduced percutaneously into the cardiovascular system of a patient via the radial, brachial or femoral artery. The catheter assembly is advanced through the coronary vasculature until the balloon portion is positioned across the occlusive lesion. Once in position across the lesion, the balloon is inflated to a predetermined size to radially compress the atherosclerotic plaque of the lesion to remodel the lumen wall. The balloon is then deflated to a smaller profile to allow the catheter to be withdrawn from the patient's vasculature.

Problems associated with the above procedure include formation of intimal flaps or tom arterial linings which can collapse and occlude the blood conduit after the balloon is deflated. Moreover, thrombosis and restenosis of the artery may develop over several months after the procedure, which may require another angioplasty procedure or a surgical by-pass operation. To reduce the partial or total occlusion of the artery by the collapse of the arterial lining and to reduce the chance of thrombosis or restenosis, a stent is implanted in the artery to keep the artery open.

Drug delivery stents have reduced the incidence of in-stent restenosis (ISR) after PCI (see, e.g., Serruys, P. W., et al., J. Am. Coll. Cardiol. 39:393-399 (2002)), which has plagued interventional cardiology for more than a decade. However, a few challenges remain in the art of drug delivery stents. For example, plaques have been associated with stenosis and restenosis. While treatments of plaque-induced stenosis and restenosis have advanced significantly over the last few decades, the morbidity and mortality associated with vascular plaques have remained significant. As another example, residues of polymer or drug in a drug delivery stent may be associated with undesirable pharmacological responses of a tissue receiving such a stent, e.g., inflammation. Therefore, there is a need for further ways of treating cardiovascular disorders associated with stenting.

The embodiments of the present invention address these and other needs.

### SUMMARY OF THE INVENTION

Provided herein is an implantable device comprising a body structure and a biosoluble coating that includes biosoluble polymer and a combination of at least one anti-proliferative agent and at least one anti-inflammatory agent. The biosoluble coating will release about 80% or more of the at least one anti-proliferative agent or at least one anti-inflammatory agent, or both, within about 1 to 21 days after deployment of an implantable device having such a biosoluble coating. The biosoluble coating can completely solvate within about one month (e.g., 30 days) after deployment of the implantable device. In some embodiments, the biosoluble coating can completely solvate within about three weeks, 15 days (e.g., two weeks), about 10 days, about one week or about 1 to 3 days after deployment of the implantable device coating. An implantable device having such a biosoluble coating becomes a bare device (e.g., bare metal stent) after the biosoluble coating including the drugs and the coating material completely dissolves or solvates. An implantable device of the present invention therefore can avail itself of the benefits of both the drug delivery system (e.g., drug delivery stent) and the bare metal system (e.g., bare metal stent). As the examples described below demonstrates, an implantable device having the biosoluble coating of invention are advantageous when compared to the current drug delivery stent systems. For example, an implantable device having a biosoluble coating of the present invention may result in less neointima thickness and have better healing.

An implantable device according to the present invention can be used to treat, prevent or diagnose various conditions or disorders. Examples of such conditions or disorders include, but are not limited to, atherosclerosis, thrombosis, restenosis, hemorrhage, vascular dissection, vascular perforation, vascular aneurysm, vulnerable plaque, chronic total occlusion, patent foramen ovale, claudication, anastomotic proliferation of vein and artificial grafts, arteriovenous anastamoses, bile duct obstruction, ureter obstruction and tumor obstruction. A portion of the implantable device or the whole device itself can be formed of the material, as described herein.

### BRIEF DESCRIPTION OF THE FIGURE

FIG. 1: Neointima—morphometric analysis graph excluding sections with processing artifact and showing granulomatous reaction on a coating including a combination of an anti-proliferative agent and an anti-inflammatory agent as illustration of the coating of present invention;

FIG. 2: % Stenosis—morphometric analysis graph excluding sections with processing artifact and showing granulomatous reaction on a coating including a combination of an anti-proliferative agent and an anti-inflammatory agent as illustration of the coating of present invention.

### DETAILED DESCRIPTION

Provided herein is an implantable device comprising a body structure and a biosoluble coating that includes biosoluble polymer and a combination of at least one anti-proliferative agent and at least one anti-inflammatory agent. The biosoluble coating will release about 80% or more of the at least one anti-proliferative agent or at least one anti-inflammatory agent, or both, within about 1 to 21 days after deployment of an implantable device having such a biosoluble coating. The biosoluble coating can completely solvate within about one month (e.g., 30 days) after deployment of the implantable device. In some embodiments, the biosoluble coating can completely solvate within about three weeks, 15 days (e.g., two weeks), about 10 days, about one week or about 1 to 3 days after deployment of the implantable device coating. An implantable device having such a biosoluble coating becomes a bare device (e.g., bare metal stent) after the biosoluble coating including the drugs and the coating material completely dissolves or solvates. An implantable device of the present invention therefore can avail itself of the benefits of both the drug delivery system (e.g., drug delivery stent) and the bare metal system (e.g., bare metal stent). As the examples described below demonstrates, an implantable device having the biosoluble coating of invention are advantageous when compared to the current drug delivery stent systems. For example, an implantable device having a biosoluble coating of the present invention may result in less neointima thickness and have better healing.

As used herein, the term “completely solvates” refers to over about 99% of the material on the biosoluble coating described herein dissolves away by the physiological fluid of a body.

As used herein, the term “agent” can be used interchangeably with the term “drug”.

As used herein, the term “biosoluble coating” refers to a coating that is soluble in the bloodstream and disappears as the drug releases.

As used herein, the term “body structure” refers to a body construct, or a portion thereof, of any implantable device. An example of such implantable device is a stent, e.g., a bare metal stent (BMS).

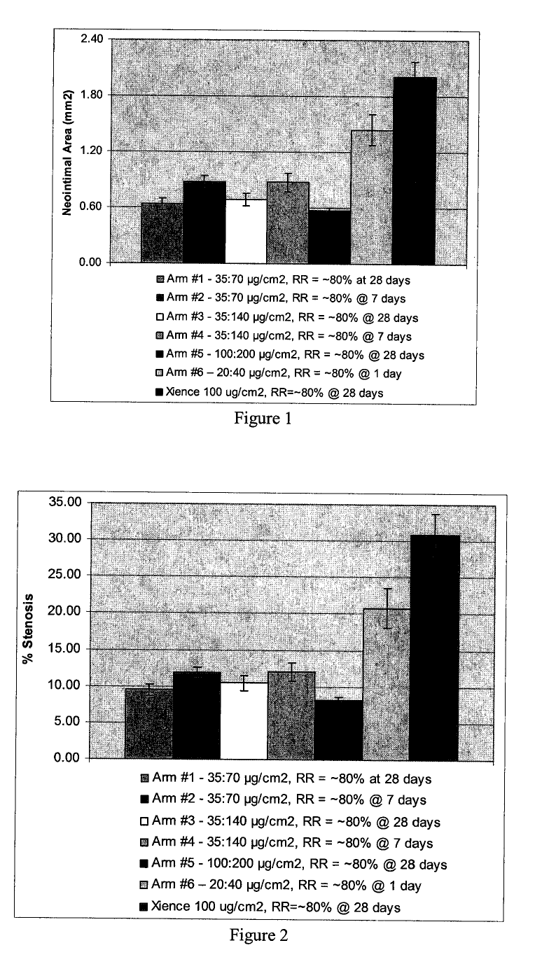

The advantages of a coating containing a combination of an anti-proliferative agent and an anti-inflammatory agent is clearly illustrated by studies using a stent having a durable coating containing zotarolimus, an anti-proliferative agent, and dexamethasone, an anti-inflammatory agent in comparison with a stent having a durable coating containing everolimus alone. As data in Tables 1 and 2 show, a coating comprising a combination of an anti-proliferative agent and an anti-inflammatory agent clearly causes faster re-endotheliazation and elicits less inflammation in a vessel tissue (see also FIGS. 1 and 2). As shown in Table 2, Arms 1-6 containing both everolimus and dexamethasone showed lower inflammation and granulomas.

TABLE 1
Histolhistologicical comparison of vessel injury and healing of
Zotarolimus:Dexamethasone-eluting Vision ™ and control
(everolimus-eluting Xience V) stents*

| | | | Mean | | | | % |
|---|---|---|---|---|---|---|---|
| Treatment | Injury | | Fibrin | Malappo | | Endothelialization | Uncovered |
| Group | Score | Fibrin (%) | Score | (%) | RBC (%) | (%) | Struts |
| A1 | 0.23 ± 0.21 | 86.89 ± 16.40 | 1.97 ± 0.35 | 0 ± 0 | 22.01 ± 16.61 | 98.91 ± 1.69 | 1.53 ± 3.10 |
| (n = 11) | | | | | | | |
| A2 | 0.17 ± 016 | 85.10 ± 21.31 | 1.70 ± 0.48 | 0.37 ± 1.11 | 24.16 ± 15.63 | 99.41 ± 1.09 | 0.37 ± 1.11 |
| (n = 9) | | | | | | | |
| A3 | 0.12 ± 0.17 | 90.37 ± 9.96 | 2.12 ± 0.62 | 0 ± 0 | 29.94 ± 16.32 | 98.12 ± 4.09 | 2.53 ± 6.38 |
| (n = 11) | | | | | | | |
| A4 | 0.12 ± 0.11 | 82.33 ± 20.70 | 1.73 ± 0.33 | 0 ± 0 | 18.11 ± 12.51 | 99.61 ± 1.09 | 0.55 ± 1.83 |
| (n = 11) | | | | | | | |
| A5 | 0.12 ± 0.083 | 70.97 ± 32.45 | 1.64 ± 0.77 | 0 ± 0 | 24.57 ± 17.80 | 85.17 ± 21.75 | 23.28 ± 32.04 |
| (n = 12) | | | | | | | |
| A6 | 0.23 ± 0.18 | 84.00 ± 15.48 | 1.56 ± 0.37 | 0 ± 0 | 17.70 ± 14.88 | 99.96 ± 0.11 | 0 ± 0 |
| (n = 9) | | | | | | | |
| Xience V | 0.17 ± 0.14 | 93.92 ± 12.44 | 1.87 ± 0.31 | 0 ± 0 | 8.20 ± 11.64 | 99.89 ± 0.26 | 0 ± 0 |
| (n = 20) | | | | | | | |
| p-value | 0.564 | 0.0802 | 0.0620 | 0.221 | 0.0061-A2, | 0.0003-A5 | <0.0001-A5 |
| | | | | | A3, A5 v. | v. Xience | v. Xience |
| | | | | | Xience | | |

*Analysis excludes sections with processing artifact

TABLE 2
Histologic comparison of inflammatory response of Zotarolimus:
Dexamethasone-eluting Vision ™ and control
(everolimus-eluting Xience V) stents*

| Treat- | | Intimal | Adventitial | |
|---|---|---|---|---|
| ment | Granulomas | Inflamm | Inflamm | Giant Cell |
| Group | (%) | Score | Score | (%) |
| A1 | 0 ± 0 | 0.18 ± 0.60 | 0 ± 0 | 4.15 ± 12.25 |
| (n = 11) | | | | |
| A2 | 0.78 ± 1.66 | 0.50 ± 0.96 | 0.067 ± 0.21 | 10.38 ± 17.00 |
| (n = 10) | | | | |
| A3 | 0 ± 0 | 0.55 ± 0.96 | 0 ± 0 | 6.65 ± 14.69 |
| (n = 11) | | | | |
| A4 | 0 ± 0 | 0.70 ± 0.84 | 0.030 ± 0.10 | 11.21 ± 16.60 |
| (n = 11) | | | | |
| A5 | 0 ± 0 | 0.19 ± 0.41 | 0 ± 0 | 1.86 ± 3.53 |
| (n = 12) | | | | |
| A6 | 1.99 ± 3.62 | 0.53 ± 0.72 | 0.50 ± 0.67 | 6.94 ± 6.46 |
| (n = 12) | | | | |
| Xience | 21.77 ± 30.86 | 1.58 ± 1.81 | 0.81 ± 0.96 | 20.04 ± 17.58 |
| V | | | | |
| (n = 37) | | | | |
| p-value | 0.0001-A1, A2, | 0.116 | <0.0001* | 0.0018-A1, A5, |
| | A3, A4, A5, | | | A6 v. Xience |
| | A6 v. Xience | | | |

*Values are expressed as the means ± standard deviation. The numbers in parenthesis correspond to the number of stent implants.

### Biosoluble Polymers

Any biosoluble polymers can be used to form a coating on a stent or to provide a drug delivery particle with the anti-proliferative drug and anti-inflammatory drug. Examples of such polymers include, but are not limited to, poly(ethylene glycol) (PEG), poly(lactide-co-glycolide)-co-poly(ethylene glycol) (PLGA-PEG) block copolymer, other PEG copolymers, poly(vinyl alcohol) (PVA), hyaluronic acid, hydroxyl cellulose, Carboxymethylcellulose (CMC), polysaccharides, phosphoryl choline containing polymers, chitosan, collagen, and combinations thereof.

### Anti-Proliferative Agents and Anti-Inflammatory Agents

In accordance with one embodiment, described herein are a drug-delivery system and the method of using the drug-delivery system. The term “treatment” includes prevention, reduction, delay or elimination of the vascular disorder. In some embodiments, treatment also includes repairing damage caused by the disorder and/or the mechanical intervention. The drug-delivery system has two or more drugs for treating a vascular disorder or a related disorder. The drugs can be a combination of at least one anti-proliferative agent, at least one anti-inflammatory agent, and optionally a third bioactive agent.

In one embodiment, the composition described herein includes an effective amount of at least one anti-inflammatory agent and an effective amount of an anti-proliferative agent. In another embodiment, the composition described herein includes an effective amount of an agent which is effective both as an anti-inflammatory agent and as an anti-proliferative agent.

### Inflammation in Stenting a Vessel

A common disorder in association with mechanical modification of a vessel, such as by a balloon or stenting, is restenosis. A number of cellular mechanisms have been proposed that lead to restenosis of a vessel. Two of these mechanisms are (1) the migration and proliferation of smooth muscle cells to and at the site of injury, and (2) the acute and chronic inflammatory response to injury and foreign body presence.

Inflammation is a defensive, biological response to injury, infection or an abrupt change in tissue homeostasis. Inflammation can occur anywhere in the body, and most of the time is confined to that part of the body. Well-known indicators of inflammation are pain, redness, warmth, swelling, and loss of function. In nature, inflammatory responses are designed to destroy, dilute and isolate injurious agents and then lead to recovery and repair of the affected tissue. The intensity of an inflammatory response can vary from one that is self-limiting, which requires minor therapeutic intervention, to one that is life threatening, which requires intense intervention. One drawback of the inflammatory process is its ability to become progressive, meaning tissue damage continues after the stimulus is neutralized or removed.

Vascular inflammation is the first stage of the inflammatory response, developing after the initial contact with the stimulus and continuing sometimes for several days. The presence of a stimulatory agent in the blood or in the tissue triggers the body's response through endothelial cells. The endothelial cell layer is the innermost layer of larger vessels and the only cell layer of the smallest vessels, the capillaries. Endothelial cells produce substances called chemokines that attract neutrophils and other white blood cells to the site of injury. Within the site, neutrophils and endothelium relay information back and forth across cell membranes through presentation of adhesion molecules and cytokines. Cellular cross-talk promotes physical interaction between the “inflamed” neutrophil and the “inflamed” endothelium.

Another important pathological feature of vascular inflammation is endothelial cell swelling. This action reduces the functional vessel diameter such that the speed of blood flow falls significantly and the vessel becomes congested. When these conditions predominate, inflamed neutrophils are induced to plug the vessel. As a result, endothelial cells lose their tight connections allowing neutrophils to transmigrate into the surrounding tissue.

Within hours of the initial stimulus, neutrophils begin to enter the tissue and may continue transmigration for many days. The appearance of inflammatory cells in the surrounding tissue marks the beginning of tissue damage. In some inflammatory conditions, tissue damage is caused by direct injury of the vessels and amplified by the subsequent recruitment of neutrophils into the tissue.

Activated by local mediators, neutrophils and tissue macrophages are triggered to release agents that destroy toxins and clean up dead cells in the area. Unfortunately, these same agents also cause collateral damage to healthy cells, which further extends the borders of the initial tissue destruction.

Tissue repair is the third and final stage of inflammation. It may take several days for tissue destruction to reach full intensity before tapering off. Until then, the tissue repair process that consists of growth of new blood vessels and entry of monocytes to clean up the debris is delayed. Fibroblasts also enter the local tissue to replace the extracellular matrix and collagen. The process of tissue repair is stringently controlled within the tissue site. If the process becomes dysregulated, inappropriate tissue repair will lead to excessive scarring. Depending on the tissue and the intensity/duration of the inflammatory condition, the amount of scarring can be significant.

An example of disorders that vessel inflammation is involved is vulnerable plaque (VP) rupture. Previous studies have demonstrated that inflammation promotes proliferation at sites of balloon angioplasty and stent placement in pigs (Komowski, et al., Coron Artery Dis. 12(6):513-5 (2001)). Since sites of vulnerable plaque have a higher density of macrophages and lymphocytes than other types of atherosclerotic lesions, it is expected that these sites, when stented, will produce elevated amounts of the cytokines (IL-1, TNF-alpha) that promote smooth muscle cell proliferation.

Another example of disorders that vessel inflammation is involved is diabetes. Studies have shown that patients with type-2 diabetes have higher rates of restenosis than the general population. The diabetic patient is in pro-inflammatory state that can amplify restenosis because diabetic lesions contain a large number of inflammatory cells (e.g., macrophages, lymphocytes, etc.).

### Anti-Proliferative Agents

Any drugs having anti-proliferative effects can be used in the present invention. The anti-proliferative agent can be a natural proteineous agent such as a cytotoxin or a synthetic molecule. Preferably, the active agents include anti-proliferative substances such as actinomycin D, or derivatives and analogs thereof (manufactured by Sigma-Aldrich 1001 West Saint Paul Avenue, Milwaukee, Wis. 53233; or COSMEGEN available from Merck) (synonyms of actinomycin D include dactinomycin, actinomycin IV, actinomycin II, actinomycin XI, and actinomycin C₁), all taxoids such as taxols, docetaxel, and paclitaxel, paclitaxel derivatives, all olimus drugs such as macrolide antibiotics, rapamycin, everolimus, structural derivatives and functional analogues of rapamycin, structural derivatives and functional analogues of everolimus, FKBP-12 mediated mTOR inhibitors, biolimus, perfenidone, prodrugs thereof, co-drugs thereof, and combinations thereof. Representative rapamycin derivatives include 40-O-(3-hydroxy)propyl-rapamycin, 40-O-[2-(2-hydroxy)ethoxy]ethyl-rapamycin, or 40-O-tetrazole-rapamycin, prodrugs thereof, co-drugs thereof, and combinations thereof.

In one embodiment, the anti-proliferative agent is everolimus. Everolimus acts by first binding to FKBP12 to form a complex (Neuhhaus, P., et al., Liver Transpl. 2001 7(6):473-84 (2001) (Review)). The everolimus/FKBP12 complex then binds to mTOR and blocks its activity (Id.). By blocking mTOR activity, cells are unable to pass through G1 of the cell cycle and as a result, proliferation is inhibited. mTOR inhibition has also been shown to inhibit vascular smooth muscle migration.

Other examples of anti-proliferative agents include, but are not limited to, midostaurin

### Anti-Inflammatory Agents

Any drugs having anti-inflammatory effects can be used in the present invention. The anti-inflammatory drug can be a steroidal anti-inflammatory agent, a nonsteroidal anti-inflammatory agent, or a combination thereof. In some embodiments, anti-inflammatory drugs include, but are not limited to, steroidal anti-inflammatory agents, a nonsteroidal anti-inflammatory agent, or a combination thereof In some embodiments, anti-inflammatory agents include clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenofibrate, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, and prodrugs thereof.

In one embodiment, the anti-inflammatory agent is clobetasol. Clobetasol is a corticosteroid that binds to corticosteroid receptors, a class of nuclear receptor. The binding of clobetasol to the corticosteroid receptor subsequently alters gene expression in such a way that inflammation is inhibited. For example, corticosteroids inhibit the activation of NFkB, the nuclear factor that is responsible for changes in gene expression that promote inflammation. The reduction in inflammation may also inhibit the mechanisms that promote small muscle cell (SMC) hyper proliferation. This is shown in that dexamethasone, a less potent glucocorticoid as compared to clobetasol, reduces the production of PGDF and thus has anti-proliferative properties. Clobetasol acts through similar pathways and is more potent than dexamethasone.

### Dosage

The dosage or concentration of the anti-proliferative and anti-inflammatory agents required to produce a favorable therapeutic effect should be less than the level at which the bioactive agent produces toxic effects and greater than the level at which non-therapeutic results are obtained. The dosage or concentration of the agents required can depend upon factors such as the particular circumstances of the patient, the nature of the trauma, the nature of the therapy desired, the time over which the ingredient administered resides at the vascular site, and if other active agents are employed, the nature and type of the substance or combination of substances. Therapeutic effective dosages can be determined empirically, for example by infusing vessels from suitable animal model systems and using immunohistochemical, fluorescent or electron microscopy methods to detect the agent and its effects, or by conducting suitable in vitro studies.

In one embodiment, the bioactive agents can be incorporated into polymeric coating in a percent loading of between about 0.01% and less than about 100% by weight, more preferably between about 5% and about 50% by weight of the total drug-load that includes greater than about 0% to about 100% of the anti-proliferative agent and less than about 100% to greater than about 0% of the anti-inflammatory agent. The relative amount of the anti-proliferative agent and anti-inflammatory agent can be determined by the type of lesions to be treated. For example, where everolimus is used as the anti-proliferative agent and clobetasol is used as the anti-inflammatory agent, the relative amount of everolimus and clobetasol can be varied for different types of lesions, that is, the relative amount of everolimus can be higher for more proliferative lesions, and on the other hand, the relative amount of clobetasol can be higher for more inflammatory lesions.

### Other Bioactive Agents

In some embodiments, other agents can be used in combination with the anti-proliferative agent and the anti-inflammatory agents. These bioactive agents can be any agent which is a therapeutic, prophylactic, or diagnostic agent. These agents can also have anti-proliferative and/or anti-inflammmatory properties or can have other properties such as antineoplastic, antiplatelet, anti-coagulant, anti-fibrin, antithrombonic, antimitotic, antibiotic, antiallergic, antioxidant as well as cystostatic agents. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of antineoplastics and/or antimitotics include methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as Angiomax ä (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of such cytostatic substance include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other therapeutic substances or agents which may be appropriate include alpha-interferon, and genetically engineered epithelial cells. The foregoing substances are listed by way of example and are not meant to be limiting. Other active agents which are currently available or that may be developed in the future are equally applicable.

### DEFINITIONS

Wherever applicable, the definitions to some terms used throughout the description of the present invention as provided below shall apply. The terms “biologically degradable” (or “biodegradable”), “biologically erodable” (or “bioerodable”), “biologically absorbable” (or “bioabsorbable”), and “biologically resorbable” (or “bioresorbable”), in reference to polymers and implantable devices, are used interchangeably and refer to polymers and implantable devices that are capable of being completely or substantially completely degraded, dissolved, and/or eroded over time when exposed to physiological conditions and can be gradually resorbed, absorbed and/or eliminated by the body, or that can be degraded into fragments that can pass through the kidney membrane of an animal (e.g., a human). The process of breaking down and eventual absorption and elimination of the polymer or implantable device can be caused by, e.g., hydrolysis, metabolic processes, oxidation, enzymatic processes, bulk or surface erosion, and the like. Conversely, wherever applicable, a “biostable” polymer or implantable device refers to a polymer or implantable device that is not biodegradable.

Whenever the reference is made to “biologically degradable,” “biologically erodable,” “biologically absorbable,” and “biologically resorbable” stent or polymers forming such stent, it is understood that after the process of degradation, erosion, absorption, and/or resorption has been completed or substantially completed, no part or substantially little part of the device will remain. Whenever the terms “degradable,” “biodegradable,” or “biologically degradable” are used in this application, they are intended to broadly include biologically degradable, biologically erodable, biologically absorbable, and biologically resorbable polymers or implantable device.

“Physiological conditions” refer to conditions to which an implant is exposed within the body of an animal (e.g., a human). Physiological conditions include, but are not limited to, “normal” body temperature for that species of animal (approximately 37° C. for a human) and an aqueous environment of physiologic ionic strength, pH and enzymes. In some cases, the body temperature of a particular animal may be above or below what would be considered “normal” body temperature for that species of animal. For example, the body temperature of a human may be above or below approximately 37° C. in certain cases. The scope of the present invention encompasses such cases where the physiological conditions (e.g., body temperature) of an animal are not considered “normal.” In the context of a blood-contacting implantable device, a “prohealing” drug or agent refers to a drug or agent that has the property that it promotes or enhances re-endothelialization of arterial lumen to promote healing of the vascular tissue.

As used herein, a “co-drug” is a drug that is administered concurrently or sequentially with another drug to achieve a particular pharmacological effect. The effect may be general or specific. The co-drug may exert an effect different from that of the other drug, or it may promote, enhance or potentiate the effect of the other drug.

As used herein, the term “prodrug” refers to an agent rendered less active by a chemical or biological moiety, which metabolizes into or undergoes in vivo hydrolysis to form a drug or an active ingredient thereof. The term “prodrug” can be used interchangeably with terms such as “proagent”, “latentiated drugs”, “bioreversible derivatives”, and “congeners”. N. J. Harper, Drug latentiation, Prog Drug Res., 4: 221-294 (1962); E. B. Roche, Design of Biopharmaceutical Properties through Prodrugs and Analogs, Washington, DC: American Pharmaceutical Association (1977); A. A. Sinkula and S. H. Yalkowsky, Rationale for design of biologically reversible drug derivatives: prodrugs, J. Pharm. Sci., 64: 181-210 (1975). Use of the term “prodrug” usually implies a covalent link between a drug and a chemical moiety, though some authors also use it to characterize some forms of salts of the active drug molecule. Although there is no strict universal definition of a prodrug itself, and the definition may vary from author to author, prodrugs can generally be defined as pharmacologically less active chemical derivatives that can be converted in vivo, enzymatically or nonenzymatically, to the active, or more active, drug molecules that exert a therapeutic, prophylactic or diagnostic effect. Sinkula and Yalkowsky, above; V. J. Stella et al., Prodrugs: “Do they have advantages in clinical practice?”, Drugs, 29: 455-473 (1985).

The terms “polymer” and “polymeric” refer to compounds that are the product of a polymerization reaction. These terms are inclusive of homopolymers (i.e., polymers obtained by polymerizing one type of monomer), copolymers (i.e., polymers obtained by polymerizing two or more different types of monomers), terpolymers, etc., including random, alternating, block, graft, dendritic, crosslinked and any other variations thereof. As used herein, the term “implantable” refers to the attribute of being implantable in a mammal (e.g., a human being or patient) that meets the mechanical, physical, chemical, biological, and pharmacological requirements of a device provided by laws and regulations of a governmental agency (e.g., the U.S. FDA) such that the device is safe and effective for use as indicated by the device. As used herein, an “implantable device” may be any suitable substrate that can be implanted in a human or non-human animal. Examples of implantable devices include, but are not limited to, self-expandable stents, balloon-expandable stents, coronary stents, peripheral stents, stent-grafts, catheters, other expandable tubular devices for various bodily lumen or orifices, grafts, vascular grafts, arterio-venous grafts, by-pass grafts, pacemakers and defibrillators, leads and electrodes for the preceding, artificial heart valves, anastomotic clips, arterial closure devices, patent foramen ovale closure devices, cerebrospinal fluid shunts, and particles (e.g., drug-eluting particles, microparticles and nanoparticles). The stents may be intended for any vessel in the body, including neurological, carotid, vein graft, coronary, aortic, renal, iliac, femoral, popliteal vasculature, and urethral passages. An implantable device can be designed for the localized delivery of a therapeutic agent. A medicated implantable device may be constructed in part, e.g., by forming the device with a material containing a therapeutic agent. The body of the device may also contain a therapeutic agent.

An implantable device can be fabricated with a material containing partially or completely a biodegradable/bioabsorbable/ bioerodable polymer, a biostable polymer, or a combination thereof. An implantable device itself can also be fabricated partially or completely from a biodegradablelbioabsorbable/bioerodable polymer, a biostable polymer, or a combination thereof. In the context of a stent, “delivery” refers to introducing and transporting the stent through a bodily lumen to a region, such as a lesion, in a vessel that requires treatment. “Deployment” corresponds to the expanding of the stent within the lumen at the treatment region. Delivery and deployment of a stent are accomplished by positioning the stent about one end of a catheter, inserting the end of the catheter through the skin into a bodily lumen, advancing the catheter in the bodily lumen to a desired treatment location, expanding the stent at the treatment location, and removing the catheter from the lumen.

### Method of Coating A Device

The coating described herein can be formed by spray coating or any other coating process available in the art. Generally, the coating involves dissolving or suspending the composition, or one or more components thereof, in a solvent or solvent mixture to form a solution, suspension, or dispersion of the composition or one or more components thereof, applying the solution or suspension to an implantable device, and removing the solvent or solvent mixture to form a coating or a layer of coating. Suspensions or dispersions of the composition described herein can be in the form of latex or emulsion of microparticles having a size between 1 nanometer and 100 microns, preferably between 1 nanometer and 10 microns. Heat and/or pressure treatment can be applied to any of the steps involved herein. In addition, if desirable, the coating described here can be subjected to further heat and/or pressure treatment. Some additional exemplary processes of coating an implantable device that may be used are described in, for example, Lambert T L, et al. Circulation, 1994, 90: 1003-1011; Hwang C W, et al. Circulation, 2001, 104: 600-605; Van der Giessen W J, et al. Circulation, 1996, 94: 1690-1697; Lincoff A M, et al. J Am Coll Cardiol 1997, 29: 808-816; Grube E. et al, J American College Cardiology Meeting, Mar. 6 2002, ACCIS2002, poster 1174-15; Grube E, et al, Circulation, 2003, 107: 1, 38-42; Bullesfeld L, et al. Z Kardiol, 2003, 92: 10, 825-832; and Tanabe K, et al. Circulation 2003, 107: 4, 559-64.

As used herein, the term “solvent” refers to a liquid substance or composition that is compatible with the polymer and is capable of dissolving or suspending the polymeric composition or one or more components thereof at a desired concentration. Representative examples of solvents include chloroform, acetone, water (buffered saline), dimethylsulfoxide (DMSO), propylene glycol monomethyl ether (PM,) iso-propylalcohol (IPA), n-propyl alcohol, methanol, ethanol, tetrahydrofuran (THF), dimethylformamide (DMF), dimethyl acetamide (DMAC), benzene, toluene, xylene, hexane, cyclohexane, heptane, octane, nonane, decane, decalin, ethyl acetate, butyl acetate, isobutyl acetate, isopropyl acetate, butanol, diacetone alcohol, benzyl alcohol, 2-butanone, cyclohexanone, dioxane, methylene chloride, carbon tetrachloride, tetrachloroethylene, tetrachloro ethane, chlorobenzene, 1,1,1-trichloroethane, 1,1,2-trichloroethane, formamide, hexafluoroisopropanol, 1,1,1-trifluoroethanol, and hexamethyl phosphoramide and a combination thereof.

Examples of such implantable devices include self-expandable stents, balloon-expandable stents, stent-grafts, grafts (e.g., aortic grafts), artificial heart valves, cerebrospinal fluid shunts, pacemaker electrodes, and endocardial leads (e.g., FINELINE and ENDOTAK, available from Guidant Corporation, Santa Clara, Calif.). The underlying structure of the device can be of virtually any design. The device can be made of a metallic material or an alloy such as, but not limited to, cobalt chromium alloy (ELGILOY), stainless steel (316L), high nitrogen stainless steel, e.g., BIODUR 108, cobalt chrome alloy L-605, “MP35N,” “MP20N,” ELASTINITE (Nitinol), tantalum, nickel-titanium alloy, platinum-iridium alloy, gold, magnesium, or combinations thereof. “MP35N” and “MP20N” are trade names for alloys of cobalt, nickel, chromium and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. Devices made from bioabsorbable or biostable polymers could also be used with the embodiments of the present invention. In one embodiment, the implantable device is a stent, which can be degradable stents, biodurable stents, depot stents, and metallic stens such as stents made of stainless steel or nitinol. The stents can be balloon expandable or self expanding.

### Method of Treating or Preventing Disorders

An implantable device according to the present invention can be used to treat, prevent or diagnose various conditions or disorders. Examples of such conditions or disorders include, but are not limited to, atherosclerosis, thrombosis, restenosis, hemorrhage, vascular dissection, vascular perforation, vascular aneurysm, vulnerable plaque, chronic total occlusion, patent foramen ovale, claudication, anastomotic proliferation of vein and artificial grafts, arteriovenous anastamoses, bile duct obstruction, ureter obstruction and tumor obstruction.

In certain embodiments, optionally in combination with one or more other embodiments described herein, the inventive method treats, prevents or diagnoses a condition or disorder selected from atherosclerosis, thrombosis, restenosis, hemorrhage, vascular dissection, vascular perforation, vascular aneurysm, vulnerable plaque, chronic total occlusion, patent foramen ovale, claudication, anastomotic proliferation of vein and artificial grafts, arteriovenous anastamoses, bile duct obstruction, ureter obstruction and tumor obstruction. In a particular embodiment, the condition or disorder is atherosclerosis, thrombosis, restenosis or vulnerable plaque.

In one embodiment of the method, optionally in combination with one or more other embodiments described herein, the implantable device can include at least one biologically active agent that is not an anti-proliferative agent or an anti-inflammatory agent. Examples of such agents are described above, which include, but are not limited to, nitric oxide donors, super oxide dismutases, super oxide dismutase mimics, 4-amino-2,2,6,6-tetramethylpiperidine-1-oxyl (4-amino-TEMPO), imatinib mesylate, estradiol, progenitor cell-capturing antibodies, prohealing drugs, prodrugs thereof, co-drugs thereof, and a combination thereof.

In certain embodiments, optionally in combination with one or more other embodiments described herein, the implantable device used in the method is selected from stents, grafts, stent-grafts, catheters, leads and electrodes, clips, shunts, closure devices, valves, and particles. In a specific embodiment, the implantable device is a stent.

### EXAMPLES

Biosoluble PLGA-PEG-PLGA polymer with various PEG content (%) is used to form exemplary coatings on 3×12 mm Vision Stents (available from Abbott Vascular, Santa Clara, Calif.) according to established procedures. The polymer is used for the primer layer as well as the drug reservoir layer. The drug reservoir layer also includes everolimus (Ever) and dexamethasone acetate (Dex). The drug to polymer ratio is D:P=1:3 (everolimus, 50 μg/cm²; dexamethasone acetate, 100 μg/cm²). Scanning electron microscope (SEM) studies show all these coatings have good mechanical properties (SEM images not shown). Total content (TC) and release rate (RR) of drugs from studies on these coatings in a 28 porcine model are summarized below in Table 3.

TABLE 3

| | TC | 1d RR | TC | |
|---|---|---|---|---|
| | Ever | Ever | Dex | 1d RR |
| Example # | n = 5 | (n = 3) | (n = 5) | Dex (n = 3) |
| 1 | 87.6 ± 2.3 | 80.7 ± 0.1 | N/A | N/A |
| PLGA-PEG-PLGA | | | | |
| (17% PEG) | | | | |
| 2 | 72.9 ± 1.3 | 100% | N/A | N/A |
| PLGA-PEG-PLGA | | | | |
| (22% PEG) | | | | |
| 3 | 63.1 ± 4.9 | 100% | 98.4 ± 1.0 | 100% |
| PLGA-PEG-PLGA | | | | |
| (22% PEG) | | | | |
| (D:P = 1:3 total) | | | | |
| 2 | 85.9 ± 1.5 | 100% | 101.3 ± 1.5 | 100% |
| PLGA-PEG-PLGA | | | | |
| (22% PEG) | | | | |
| (D:P = 1:3 Ever) | | | | |

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. An implantable device, comprising a biosoluble coating deposited upon a body structure, the coating comprising:
a combination of an anti-inflammatory drug and an anti-proliferate drug, and
a biosoluble polymer for control the release of the anti-inflammatory drug and the anti-proliferate drug from the coating such that about 80% of the anti-inflammatory drug and/or about 80% of the anti-proliferate drug releases from the coating within about 1 to 3 days after deployment of the implantable device,
wherein the biosoluble coating solvates completely within about 30 days after deployment of the implantable device.

2. The implantable device of claim 1, wherein about 80% of the anti-inflammatory drug and about 80% of the anti-proliferate drug release from the coating within about 1 to 3 days after deployment of the implantable device.

3. The implantable device of claim 1, wherein about 80% of the anti-inflammatory drug and about 80% of the anti-proliferate drug release from the coating within about 1 to 21 days after deployment of the implantable device.

4. The implantable device of claim 1, wherein the biosoluble coating solvates completely within about 21 days after deployment of the implantable device.

5. The implantable device of claim 1, wherein the biosoluble coating solvates completely within about 14 days after deployment of the implantable device.

6. The implantable device of claim 1, wherein the biosoluble coating solvates completely within about 7 days after deployment of the implantable device.

7. The implantable device of claim 1, wherein the biosoluble coating solvates completely within about 1 to 3 days after deployment of the implantable device.

8. The implantable device of claim 1, wherein the biosoluble polymer is selected from poly(ethylene glycol) (PEG), poly(lactide-co-glycolide)-co-poly(ethylene glycol) (PLGA-PEG) block copolymer, other PEG copolymers, poly(vinyl alcohol) (PVA), hyaluronic acid, hydroxyl cellulose, Carboxymethylcellulose (CMC), polysaccharides, phosphoryl choline containing polymers, chitosan, collagen, and combinations thereof.

9. The implantable device of claim 1, wherein the anti-proliferative agent is selected from the group consisting of taxoids, everolimus, rapamycin, derivatives of everolimus, derivatives of rapamycin, derivatives of taxoids, and combinations thereof.

10. The implantable device of claim 1, wherein the anti-inflammatory agent is clobetasol.

11. The implantable device of claim 1, wherein the body structure is a bare metal stent.

12. A method of fabricating an implantable device, comprising
depositing on the implantable device a biosoluble coating onto a body structure, the biosoluble coating comprising
(a) a combination of an anti-inflammatory drug and an anti-proliferate drug, and
(b) a biosoluble polymer for control the release of the anti-inflammatory drug and the anti-proliferate drug from the coating such that about 80% of the anti-inflammatory drug and/or about 80% of the anti-proliferate drug releases from the coating within about 1 to 3 days after deployment of the implantable device,
wherein the biosoluble coating solvates completely within about 30 days after deployment of the implantable device.

13. The method of claim 12, wherein about 80% of the anti-inflammatory drug and about 80% of the anti-proliferate drug release from the coating within about 1 to 3 days after deployment of the implantable device.

14. The method of claim 12, wherein about 80% of the anti-inflammatory drug and about 80% of the anti-proliferate drug release from the coating within about 1 to 21 days after deployment of the implantable device.

15. The method of claim 12, wherein the biosoluble coating solvates completely within about 21 days after deployment of the implantable device.

16. The method of claim 12, wherein the biosoluble coating solvates completely within about 14 days after deployment of the implantable device.

17. The method of claim 12, wherein the biosoluble coating solvates completely within about 7 days after deployment of the implantable device.

18. The method of claim 12, wherein the biosoluble coating solvates completely within about 1 to 3 days after deployment of the implantable device.

19. The method of claim 12, wherein the biosoluble polymer is selected from poly(ethylene glycol) (PEG), poly(lactide-co-glycolide)-co-poly(ethylene glycol) (PLGA-PEG) block copolymer, other PEG copolymers, poly(vinyl alcohol) (PVA), hyaluronic acid, hydroxyl cellulose, Carboxymethylcellulose (CMC), polysaccharides, phosphoryl choline containing polymers, chitosan, collagen, and combinations thereof.

20. The method of claim 12, wherein the anti-proliferative agent is selected from the group consisting of taxoids, everolimus, rapamycin, derivatives of everolimus, derivatives of rapamycin, derivatives of taxoids, and combinations thereof.

21. The method of claim 12, wherein the anti-inflammatory agent is clobetasol.

22. The method of claim 12, wherein the body structure is a bare metal stent.

23. A method of a medical condition in a blood vessel comprising implanting in the blood vessel an implantable device according to claim 1.

24. A method of a medical condition in a blood vessel comprising implanting in the blood vessel an implantable device according to claim 11.
